(19) United States
(12) Patent Application Publication
(10) Pub. No.: US 2022/0010390 A1
(43) Pub. Date: Jan. 13, 2022

(54) Method for utilizing biomasses

(71) Applicant: Oy Karl Fazer Ab, Helsinki (FI)

(72) Inventors: Anna Nicol, Helsinki (FI); Janne Toivanen, Helsinki (FI)

(21) Appl. No.: 17/428,999

(22) PCT Filed: Feb. 7, 2020
(86) PCT No.: PCT/FI2020/050075

§ 371 Date: Aug. 6, 2021

(30) Foreign Application Priority Data
Feb. 7, 2019 (FI) 20195087

(51) Int. Cl. C13K 13/00; C12P 19/02; C10L 5/44; C12P 19/14

(52) U.S. Cl. CPC C13K 13/002; C12P 19/14; C10L 5/445; C12P 19/02

(57) ABSTRACT

The present invention relates to utilization of bio-based materials. In particular, the present invention relates to a method for utilization of grain based raw materials, wherein the method also comprises a versatile use of the side streams or by-products of the process. The method produces a wet fiber cake for combustion, wherein said wet fiber cake provides excellent burn values, low emission values and low amount of residual ash.

### FIELD

The present invention relates to utilization of bio-based materials, more particularly grain based materials. In particular, the present invention relates to a method for utilization of grain cultivation by-products, more specifically oat hulls, wherein the method also comprises a versatile use of the side streams or by-products of the process. After hydrolysis of the bio-based material, the method of the invention produces a wet fiber cake for combustion, with excellent burn value, low emission values and low amount of residual ash.

### BACKGROUND

Different biomass has been identified as a good source for extraction and utilization of various carbohydrate products, including D-Xylose. Whilst the extraction and purification of carbohydrates, including sucrose and D-Xylose, is often technically feasible, the commercial implementation of such processes is often deemed not commercially viable due to difficulties in dealing with various solid and liquid by-product fractions deriving from such processes, as well as high CAPEX (capital expenditure) needs associated with such processes.

The commercial production of sucrose from sugar beet and sugar cane highly depends on utilization of the by-product streams for other applications including animal feed, ethanol production, as well as burning the residual solid fractions for renewable energy. In case of D-Xylose from biomass, this approach is not self-evident due to the nature of the by-products derived from such processes, as well as difficulty of producing a good enough solid biomass by-product fraction following extraction of carbohydrates for an efficient and commercially viable combustion process.

The use of grain cultivation by-products, such as oat hulls, for carbohydrate production has been studied in the past, but commercial implementation has not been viable to date due to aforementioned difficulties, which also include logistic handling of vast amounts of solid biomass by-product from such processes which is challenging to collect, treat, and burn for energy.

Several power plants produce energy (stream, electricity, and heat) by combustion of various biomass waste materials. However, in case of producing various carbohydrates from biomass, the nature of the solid biomass fraction following extraction of carbohydrates has prevented achieving an efficient and commercially viable combustion process with low emissions of impurities. For example, the contents of moisture, salts, and sugars in the solid biomass fraction are parameters, which influence the operation of combustion boilers.

U.S. Pat. No. 4,612,286 discloses a method of producing fuel alcohol from biomass, wherein the method comprises acid hydrolysis, fermentation of pentose and hexose sugars of the hydrolysate to produce fuel alcohol, as well as washing, dewatering, and burning the biomass to produce energy for the process. WO 2014/138535 A1 discloses systems for separating solids from liquids of saccharified biomass material slurries to produce useful products, such as fuels. WO 2015/081439 A1 relates to a process for enzymatic hydrolysis of a pretreated lignocellulosic feedstock, wherein the hydrolysing step is conducted in the presence of a polymer.

Wet combustion of bagasse in sugar factory boilers has been used for production of energy for the process. According to one study, wet bagasse comprises approximately 50% moisture (Abdalla et al, 2018) but the burning results indicated that to obtain reasonable evaporation coefficient during wet bagasse burning, bagasse should contain moisture ranges 45-37% per unit mass of bagasse.

CA 639458 discloses wet combustion of sulphite waste liquor wherein the combustion gas leaving the reactor is cooled sufficiently to cause steam contained therein to condense and using the combustion gas to drive a gas turbine. Miccio et al (2014) studied fluidized bed combustion of wet biomass fuel (olive husks) and found that olive husks having a water content between 60 and 70% by mass can be burnt in fluidized bed in a bed temperature range between 800 and 850° C.

However, a problem of prior art processes utilizing various biomass feedstocks for the production of carbohydrates has been how to efficiently utilize the by-products and side streams generated in the process. Also new lignocellulosic raw biomasses that could find an alternative use, instead of being considered as waste materials, are needed. Moreover, if higher burn values and lower emissions could be achieved in the combustion of biomass waste materials, particularly in wet combustion processes, this would advance the use of said materials as sources of energy.

Thus there is still a need for a process for utilizing bio-based materials, particularly a need for an improved method for producing various carbohydrate products, which efficiently uses renewable resources and utilizes the by-products and side streams of the process in an economically and environmentally sustainable way.

### SUMMARY OF THE INVENTION

The invention is defined by the features of the independent claims. Some specific embodiments are defined in the dependent claims.

The present invention is based on the finding of an efficient overall process concept for utilization of bio-based materials, particularly grain cultivation by-products, and more particularly oat hulls. From lignocellulosic biomasses the method produces a liquid fraction, which is rich in carbohydrates, in particular D-Xylose, and a solid fraction for use as an energy source by combustion. The present invention comprises hydrolysis to obtain a hydrolysate, which is separated to a liquid fraction and to a solid fraction, comprising mainly insoluble fibres of the biomass. The solid fraction is washed and optionally pressed to obtain a low-salt and low-sugar, wet fiber cake, which is recovered and used as an energy source by combustion, particularly by wet combustion. The carbohydrate-containing liquid fraction is recovered and can be used in the production of various carbohydrate products, for example D-xylose and xylitol.

According to a first aspect of the present invention, there is thus provided a method for utilizing oat hulls, wherein the method comprises the steps of hydrolysing the oat hulls to obtain a hydrolysate; separating the hydrolysate to a liquid fraction and to a solid fraction; washing and optionally pressing the solid fraction to obtain a wet fiber cake having a DS content of 40 to 90%; recovering the liquid fraction to obtain a carbohydrate-containing liquid fraction; recovering the wet fiber cake, and utilizing it as an energy source by combustion.

Another object of the present invention is a low-salt and low-sugar, wet fiber cake having a DS content of 40 to 90%, in particular 40-85%, 40-60%, 40-50%, 50-85%, 50-70%, 60-70%, or approximately 60-65%, wherein said wet fiber cake is obtained from the hydrolysis of oat hulls, and contains an amount of salts, which is at least 20% smaller on a dry weight basis than that of the starting material.

A further object of the invention is a low-salt and low-sugar, wet fiber cake obtainable by the method of the invention.

Considerable advantages are obtained by the invention. First, the method of the invention provides use to otherwise discarded or only partly utilized biomass materials, namely oat hulls. The invention also provides an efficient industrial process for utilization of oat hulls that can provide various carbohydrate products whilst utilizing also the side streams generated in the process.

In particular, the invention provides an energy source that can be directly used in combustion with high fuel values and cleanliness properties, as will be explained in more detail in the detailed description. Combustion of the wet fiber cake obtained by the method of the present invention provides lower emissions and a lower amount of residual ash compared to combustion of the same starting material which has not been processed according to the present invention.

When oat hulls are used as a raw material to produce a carbohydrate-rich liquid extract, the residual non-dissolved cake is separated, for example by filtration, and washed to give a solid fiber cake with minimum 40% dry solids and low mineral content which can be combusted as such or mixed with dry fractions of other bio-based materials.

Further features and advantages of the present technology will appear from the following description of some embodiments.

### EMBODIMENTS

### Definitions

In the present context, the terms “grain cultivation by-products” and “grain residuals” comprise byproducts from processing of grains and parts thereof. The term thus comprises for example oat, barley, wheat, rye, and parts thereof, such as cobs, husks, hulls, leaves, or straw, in particular husks, hulls, or straw, in particular oat hulls, wheat hulls, and rye hulls, more particularly oat hulls.

The terms “oat hulls” or “oat husks” refer to outer envelopes of the oat grain (Avena sativa L.). Oat hulls are by-products of oat processing, typically obtained by mechanical separation of the hulls from the kernels prior to milling. The mechanical separation of hulls and kernels can be accomplished for example by using a rotating drum, followed by air aspiration to separate the hull fraction from the groats, i.e. the edible huskless grains. In the present method, oat hulls may be used as such or in ground or milled form.

Within this disclosure, the term “low-salt” refers to products, wherein the amount of salts is at least 20%, preferably at least 30% lower than the amount of said components in the starting bio-based material, on a dry weight basis.

“Salts” are ionic compounds that dissociate in water. These salts can include both organic and inorganic salts.

“Sugar” in the term “low-sugar” refers to soluble carbohydrates, particularly mono- and disaccharides, including glucose, fructose, xylose, xylobiose, sucrose, galactose, arabinose, and trehalose.

Within this disclosure, a reference to an “essentially hemicellulose-free” substance or composition means that said substance or composition contains no more than 5%, preferably no more than 4%, 3%, 2%, or 1% of hemicellulose, on a dry weight basis.

It has been found that an efficient overall process concept for utilization of biomasses and for the production various carbohydrate products, including D-Xylose and optionally xylitol, can be based on grain materials, particularly parts of grain, such as grain hulls, husks or straw, more particularly oat hulls. The method of the invention comprises at least the steps of hydrolyzing the biomass; separating the hydrolysate to a solid fraction and to a liquid fraction; washing, and optionally pressing the solid fraction to obtain a low-salt, wet fiber cake; recovering the carbohydrate-containing liquid fraction and optionally passing it to production of carbohydrates; recovering the wet fiber cake and utilizing it as an energy source by combustion.

In one embodiment, the invention relates to a method producing a wet fiber cake for combustion, wherein the method comprises the aforementioned steps, starting from oat hulls.

In a preferred embodiment, the process comprises at least one pressing step.

In an embodiment, the lignocellulosic biomass comprises grain parts, particularly grain hulls, such as oat, barley, wheat or rye hulls, in particular oat hulls.

Traditionally, oat has been mainly used for animal feed manufacture. However, it is increasingly used for human consumption due to its health promoting properties. Typical for oat is that the kernel is surrounded by hulls, which must be mechanically separated before the kernel goes to milling steps. Some 25-30% of the grain harvest is hulls. Oat hulls are rich in non-soluble fibers and contain 30-35% of its dry weight both cellulose and hemicellulose. Oat hull hemicellulose is a very rich source of D-xylose (70-80%) with high D-xylose/L-arabinose ratio (8-12). Typical dry matter content of hulls is 90%. Oat hulls contain very little (<5%) of both starch and protein and thus it is important to find valuable applications for hulls.

Lignin content of oat hulls is claimed to vary between 2 and 10% of DS (Welch et al. 1983), which is very low compared to for example tree biomasses and only ca. 10% of that is acid soluble. Lignin has high burning energy value and disturbs processing if present in the liquor. Thus in the production of sugar-rich liquors for different applications and solid fiber material as a side-stream for energy production it is advantageous to have as much of the lignin as possible in the solid fiber side-stream. Typical ash content of oat hulls is 4% of DS.

Hydrolysis of Biomass

Hydrolysis of the biomass material, namely oat hulls, is carried out to free sugars, in particular D-xylose, from the lignocellulosic material. Oat hulls contain D-xylose in xylan polymer form, which in the hydrolysis is broken down to release and extract D-xylose from said material. In one embodiment of the invention, the hydrolysis conditions are optimized to maximize the D-xylose yield.

Typically, hydrolysis is carried out with acids. However, it is also possible to first extract the biomass with water, preferably pressurized hot water, followed by enzymatic hydrolysis of the solubilized oligo- and polysaccharides including those comprising of D-xylose. Hydrolysis may also be carried out by employing high pressure aqueous hydrolysis by means of steam explosion.

The acids for use in the acid hydrolysis can be selected from acids capable to catalyse the conversion of hemicellulose to respective sugars. Such acids include but are not limited to sulphuric acid, hydrochloric acid, nitric acid, and phosphoric acid. Typically, sulphuric acid is used. Sulphite cooking is also used as a means to hydrolyse biomass in industrial scale.

Concentration of the acid during hydrolysis may vary depending on the conditions and the grain based material. Typically, sulphuric acid concentration of at least 0.2%, more preferably at least 0.5% to about 1% by weight of the reaction mass, is sufficient for grain based materials, such as oat hulls, when grain material loading net weight is 20% of the reaction mass.

Typically, acid hydrolysis is carried out at a temperature of 100 to 160° C., for example at about 140° C., under pressure of 3 to 4 bar, such as about 3.6 bar. The reaction time required to hydrolyse the grain based material under said temperature and pressure conditions is usually about 30-180 min, preferably about 60-90 min.

After acid hydrolysis, pH of the reaction mass is adjusted to a pH of around 1 to 7, typically around pH 3 to 5, with a base, such as NaOH. If desired, the pH adjustment step can be repeated. The hydrolysate is in the form of a slurry, where part of the grain based material has been dissolved into the liquid phase, which typically contains sugars, salts, organic acids and lignin. The solid phase comprises solid fiber from the grain based material, such as cellulose and acid-insoluble lignin.

When the grain material comprises or is oat hulls, the liquid phase typically contains D-Xylose, other sugars, salts, organic acids, and lignin. In an embodiment, where the starting material is oat hulls, approximately 25 to 40%, roughly 30-35%, of the hull mass is in the liquid phase and approximately 60 to 75%, such as about 65% as solid fiber.

Separation and Pre-Treatment

After hydrolysis, the solid fiber is separated and recovered from the hydrolysate slurry to obtain a wet fiber cake. The liquid fraction is recovered and forwarded to optional purification and recovery process of D-Xylose and other carbohydrates.

Separation of the hydrolysate into a liquid fraction and to a solid fraction can be accomplished by any suitable separation technique, such as by filtering, centrifuging, or pressing the hydrolysate to a liquid fraction and to a solid fiber fraction, for example by pressure or vacuum filters. Another means to separate the solid fraction from liquid fraction is to employ hydrocyclones. The method of the invention may comprise one or more separation steps. In case of several separation steps, different separation techniques or their combinations can be used.

Typically, the pH adjusted hydrolysate is filtrated to separate the solids and the liquid, for example by a pressure filter. Filtration may comprise one or several filtration steps, for example a primary filtration step and, if necessary, also a second filtration step (fine filtration step). In an embodiment, the first filtration step may remove even 99% of the solids, which are recovered. If desired, the required filtrate can be concentrated to a suitable dry solids content for example by evaporation.

After the first filtration, the carbohydrate-containing liquid fraction typically still contains a small amount of solids. Depending on the subsequent use, the liquid fraction may be used as such or subjected to a second filtration. The aim of the second filtration is to remove virtually all remaining solids from the D-Xylose containing liquid.

According to an embodiment, the method of the invention thus comprises at least one filtration step, preferably a first filtration step, which separates at least 80% of the solids of the hydrolysate to the solid fraction and provides a carbohydrate-rich liquid fraction, and a second filtration step, wherein essentially all remaining solids, particularly over 99% of the total solids, are removed from the carbohydrate-rich liquid fraction.

The method of the invention comprises at least one washing step to obtain a low-salt, wet fiber cake. Said washing step can be included in the step of separating the hydrolysate into a solid fraction and to a liquid fraction or in subsequent process steps. When separation is carried out by filtration, a washing step is preferably included in the filtration step. In case of two filtration steps, a washing step is preferably included in the latter washing step.

A washing step may also be included between two separation steps or between a separation step and a separation/pressing step.

Utilization of the Solid Fraction from Hydrolysis

The separated solid fraction, i.e. the solid fiber cake, contains most if not all of the cellulose of the oat hulls. However, it is essentially free of hemicellulose. This solid fraction of the hydrolysis can be converted to energy by direct combustion or by using it for example in the production of biofuels, such as bioethanol. It has surprisingly been found out that the solid fraction, particularly the low-salt and low-sugar, high DS wet fiber cake from hydrolysis of oat hulls, provides an energy source with high burn values and excellent cleanliness properties.

Other options for utilization of the solid fraction include for example its use as raw material of biodegradable packaging materials, as binding material, in ethanol production, as a fibre source for human and animal food/feed, or as a substrate for mushroom cultivation.

Combustion of the Wet Fiber Cake

The solid fraction that contains mainly insoluble fibres is washed with water to remove at least part of remaining salts, sugars, and minerals, and optionally pressed to obtain a low-salt, wet fiber cake having a dry solids content of 40 to 90%. Washing is preferably included in the separation step, preferably in a filtration step, as explained above. In an embodiment, the wet fiber cake has a DS content of 40-85%, 40-60%, 40-50%, 50-85%, 50-70%, 60-70%, or approximately 60-65%.

In an embodiment, the resulting wet fiber cake contains at least 20%, preferably at least 30%, less salts than the oat hull starting material, on a dry matter basis.

In an embodiment, the resulting wet fiber cake is essentially hemicellulose-free or contains no more than 5%, 4%, 3%, 2% or 1% hemicellulose, on a dry matter basis.

In a further embodiment, the resulting wet fiber cake contains at least 20% less salts than the oat hull starting material, on a dry matter basis, and is essentially hemicellulose-free.

The undried high DS fiber cake has good burn properties and can be used as such in combustion. It has excellent burn values, which is shown by higher gross and net calorimetric values in comparison to corresponding biomass, which has not been processed according to the present method. Further, the wet fiber cake obtained by the present method has good cleanliness properties when combusted, i.e. it provides low emissions of impurities, such as NOx, and a low amount of residual ash. Overall, the wet fiber cake obtained in the present process improves combustion efficiency by providing a cleaner combustion process, which also means less cleaning shutdowns and longer cleaning intervals.

In a further embodiment, the wet fiber cake having a dry solids content of 40 to 70% may be mixed with dry fractions of other (waste) materials, preferably waste materials from grain processing, such as grain hulls or straw. In a mixture comprising the wet fiber cake from oat hull hydrolysis and dry side stream particles from oat hull processing, the mixture preferably has a dry solids content of 70-80%.

In an embodiment, the wet fiber cake, which has a DS content of 40-70% is thus mixed with dry fractions of bio-based materials, particularly with dry hulls or dry fine solids (dust) of grains, such as dry grain hulls from grain milling, particularly from oat, wheat, and rye milling, before combustion.

In a further embodiment, the high DS wet fiber cake is mixed with said dry fractions of bio-based materials to obtain a mixture of the high DS wet fiber cake and the dry fractions of bio-based materials, wherein the mixture has a DS content of 50-85%, preferably 70-80%. The high DS wet fiber cake is preferably mixed with dry fine solids from grain milling, such as oat, wheat, and rye milling, before combustion.

The use of a high DS wet fiber cake (dry solids 40-70%) enables to mix other waste materials to the cake to provide a good mixture for combustion. As a result, fuel content meaning energy, solids, and other particles is homogenous to be processed in combustion process. Also fuel handling and combustion process controllability are improved. This allows also compressing the mixed fuel to pellets or briquettes or other type of compressed formats, if fuel needs to be transported, stored or used in combustion technology, where a pressed format is needed due to combustion, transportation or other reasons.

A high DS wet fiber cake, which has a lowered content of sugar, salts and other particles, will decrease or remove possibility of ash sintering, thus improving combustion process efficiency and controllability. Ash from the combustion of the wet fiber cake obtained by the method of the invention typically has a higher melting point than ashes from combustion of agrobiomasses. Mixing the wet fiber cake with other residuals allows better burning/combustion controllability of the fuel in the combustion chamber and in this way temperatures, particles, ash and emissions through the burning process can be controlled more easily. This will also increase the life time of combustion process equipment.

The undried fiber cake can be burned using any combustion technology applicable to solid fuels, including but not limited to fluidized bed combustion (FBC) and grate boilers. Usually, material with larger granular or particle size can be burned in both FBC boilers and grate boilers, while fine dry solids (dust and powder) are often burned only in grate boilers, by blowing them into secondary air. Moreover, handling and processing of fine dry solid material from grain processing, such as dry non-processed grain hulls, is challenging in any combustion technology.

In the present invention, however, where wet fiber cake from grain processing, particularly from oat hull processing, is burned in an FBC boiler or a grate boiler, also fine dry solids can be included in the fuel stream. The moisture of the undried fiber cake binds the fine dry solids, thus preventing them from floating in the air and avoiding any adverse effects on the filters and boilers of the power plant. Moreover, as floatable dry fine solids are explosive and catch fire easily, combustion of the same with the undried fiber cake increases industrial safety.

The residual ash from the combustion of wet fiber cake can be recovered and utilized for example as fertilizers, as components in the manufacture of building products, in feed additives, or as a source of silica, for example in the manufacture of silicon carbide. Typically, combustion of the fiber cake obtained by the present method from oat hulls produces ash, which has a high purity and can be used as such in the afore mentioned applications.

Moreover, the absolute amount of residual ash is smaller than in combustion of for example dry hull pellets, due to the method of the present invention, which produces a cleaner fiber cake for combustion than the processes of the prior art.

Use of Liquid Fraction with or without Separation

The liquid fraction from hydrolysis of oat hulls is rich on carbohydrates. It contains mainly D-Xylose in addition to other sugars, salts, organic acids and lignin. After one or more, typically two, filtration steps the liquid fraction may be concentrated to a desired DS content and passed for example to the production of carbohydrates, such as D-xylose.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

### EXPERIMENTAL

### Example 1

Oat hulls from mill operations are suspended in water containing 1% of sulphuric acid. The liquor is heated under pressure to 135° C. and agitated for 60 minutes. After the hydrolysis/extraction the mass is cooled to 70° C. and neutralized to pH 4 by adding NaOH. After pH adjustment the slurry is filtrated and the filter cake washed with water to obtain a high purity D-Xylose liquor. The solid filter cake is pressed to 55% Dry Solids cake, which is taken to combustion for energy and subsequent collection of residual ash for use as a mineral additive.

### Example 2. Results from Combustion Experiments

A wet fiber cake obtained according to Example 1 by using oat hulls as starting material was burned in a laboratory test oven at a temperature of 550° C. Heat values, emissions and the amount of residual ash were analyzed. For comparison, the same analysis was carried out from combustion of pellets made of dry oat hulls. Results are shown in Table 1. The amounts are expressed on a dry matter basis.

TABLE 1
Results from combustion tests

| | Oat hull pellets | Oat hull fiber cake |
|---|---|---|
| Residual ash, m-% | 5.6 | 4.5 |
| Sulphur, m-% | 0.08 | 0.08 |
| Gross calorimetric value, MJ/kg | 18.73 | 19.60 |
| Net calorimetric value, MJ/kg | 17.43 | 18.36 |
| Net calorimetric value, MWh/t | 4.84 | 5.1 |
| C, m-% | 46.7 | 48.7 |
| H, m-% | 6.0 | 5.7 |
| N, m-% | 0.83 | 0.59 |
| Cl, m-% | 0.075 | 0.005 |
| Na, mg/kg | 150 | 48 |
| K, mg/kg | 6000 | 96 |

The results show that the amount of residual ash is about 20% smaller after combustion of the fiber cake obtained by the method of the present invention. It is particularly noteworthy that the gross calorimetric value and the net calorimetric value show increased heat values for the fiber cake produced by the method of the invention.

The amount of sulphur is close to the same level, which is a good result taking into account that in Example 1 the oat hulls were extracted with sulphuric acid. The amount of nitrogen is smaller, while hydrogen is approximately at the same level, and carbon content is slightly higher. The fiber cake obtained by the method of the present invention also produces lower Cl, Na, and K amounts compared to a dry pellet prepared from the same starting material.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in utilization of biomasses, for example in production of various carbohydrate products, including D-xylose, from grain biomasses, wherein particularly the by-products and side streams of the process are utilized in an economically and environmentally sustainable way in energy production. In particular, the method of the invention produces a wet fiber cake for combustion, wherein said wet fiber cake provides excellent burn values, low emission values and a low amount of residual ash.

### CITATION LIST

### Patent Literature

U.S. Pat. No. 4,612,286
WO 2014/138535 A1
WO 2015/081439 A1

### Non Patent Literature

Abdalla et al, 2018, Performance of Wet and Dry Bagasse combustion in Assalaya Sugar Factory, Sudan Innov Ener Res 7:179.
Miccio et al, 2014, Fluidized Bed Combustion of Wet Biomass Fuel (Olive Husks), Chemical Engineering Transactions, Vol 37, 1-6.
Welch et al. 1983, The composition of oat husks and its variation due to genetic and other factors. J. Sci. Food Agric. Vol 34, 417-426.

1. A method for utilizing oat hulls, comprising the steps of:
hydrolysing the oat hulls to obtain a hydrolysate;
separating the hydrolysate to a liquid fraction and to a solid fraction;
washing and optionally pressing the solid fraction to obtain a wet fiber cake having a DS (dry solids) content of 40 to 90%;
recovering the liquid fraction to obtain a carbohydrate-containing liquid fraction;
recovering the wet fiber cake; and
utilizing the wet fiber cake as an energy source by combustion.

2. The method according to claim 1, wherein the wet fiber cake has a DS content of 50-70%.

3. The method according to claim 1, wherein the hydrolysis is carried out by employing acid hydrolysis, followed by pH adjustment with a base.

4. The method according to claim 1, wherein the wet fiber cake is washed with water before the recovering the wet fiber cake.

5. The method according to claim 1, wherein the wet fiber cake contains at least 20% less salts than the oat hull starting material, on a dry matter basis.

6. The method according to claim 1, wherein the wet fiber cake is essentially hemicellulose-free.

7. The method according to claim 1, wherein the wet fiber cake is mixed with dry fractions of dry hulls or dry fine solids of grains before combustion.

8. The method according to claim 7, wherein the wet fiber cake is mixed with said dry fractions to obtain a mixture of the wet fiber cake and the dry fractions, wherein the mixture has a DS content of 50-85%.

9. The method according to claim 7, wherein the wet fiber cake is mixed with dry fine solids from oat, wheat, or rye milling before combustion, and wherein the combustion comprises fluidized bed combustion.

10. The method according to claim 1, wherein the hydrolysis is carried out by first extracting the oat hulls with water, followed by enzymatic hydrolysis.

11. The method according to claim 1, wherein the separation of the hydrolysate to a liquid fraction and to a solid fraction comprises at least one filtration step, which separates at least 80% of the solids of the hydrolysate to the solid fraction and provides the carbohydrate rich liquid fraction, and a second filtration step, wherein over 99% of the total solids, are removed from the liquid fraction.

12. The method according to claim 1, wherein the separation of the hydrolysate to a liquid fraction and to a solid fraction comprises at least one filtration step, which also includes washing the solid fraction to obtain the wet fiber cake.

13. The method according to claim 1, wherein a washing step is included between two separation steps or between a separation step and a pressing step.

14. The method according to claim 7, wherein residual ash from the combustion of the wet fiber cake or from the combustion of the mixture of the wet fiber cake and the dry fractions of dry hulls or dry fine solids of grains is recovered and utilized as a fertilizer, as a component in the manufacture of building products, as a feed additive, or as a source of silica.

15. The method according to claim 1, wherein the carbohydrate-containing liquid fraction from the hydrolysis of oat hulls is utilized in the production of carbohydrates.

16. A wet fiber cake having a DS content of 40 to 90%, wherein said wet fiber cake is derived from hydrolysis of oat hulls, and contains an amount of salts, which is at least 20% smaller than that of the starting material, on a dry weight basis.

17. The wet fiber cake according to claim 16, which comprises no more than 5% hemicellulose on a dry weight basis.

18. A wet fiber cake obtainable by the method according to claim 1 having a DS content of 40 to 90% and an amount of salts, which is at least 20% smaller, on a dry matter basis, than that of the oat hulls.

19. The wet fiber cake of claim 18, wherein said wet fiber cake is essentially hemicellulose-free.

20. The method according to claim 1, wherein the carbohydrate-containing liquid fraction from the hydrolysis of oat hulls is utilized in the production of D-xylose and xylitol.
